(19) United States
(12) Patent Application Publication
(10) Pub. No.: US 2003/0171340 A1
(43) Pub. Date: Sep. 11, 2003

(54) Methods of disease treatment using metal-complexed tetracycline antibiotics

(76) Inventor: Jenefir Isbister, Potomac, MD (US)

Correspondence Address: CARELLA, BYRNE, BAIN, GILFILLAN,, CECCHI, STEWART & OLSTEIN, 6 Becker Farm Road, Roseland, NJ, 07068, US

(21) Appl. No.: 10/359,388

(22) Filed: Feb. 5, 2003

Related U.S. Application Data
(60) Provisional application No. 60/355,560, filed on Feb. 7, 2002.

(51) Int. Cl. A61K 31/65

(52) U.S. Cl. 514/152

(57) ABSTRACT

A method for treating bacterial diseases, including bacterial infections, that are otherwise resistant to antibiotics, such as tetracycline and related compounds, using metal-complexed antibiotics is disclosed. Also disclosed is a method for protecting against such diseases. The metal-complexed antibiotics include tetracyclines complexed with metals such as iron, copper and calcium.

This application claims priority of U.S. Provisional Application Serial No. 60/355,560, filed Feb. 7, 2002, the disclosure of which is hereby incorporated by reference in its entirety.

### FIELD OF THE INVENTION

This invention relates to a method of treating antibiotic, especially cycline and quinolone antibiotic, resistant bacterial infections using metal complexed cyclic antibiotics, such as metal-complexed tetracycline.

### BACKGROUND OF THE INVENTION

A wide variety of antibiotics have been used to combat bacterial infection while the development of antibiotic resistance continues to increase. The latter problem has been largely the result of both misuse and overuse of antibiotics and therapeutic agents, which serves to select for microorganisms carrying the relatively rare trait(s) providing resistance to these same antibiotics. This selection method results in a higher frequency of the traits providing resistance and a population of antibiotic resistant microorganisms. Most resistance determinants can be transferred to other bacteria via plasmids and transposons exchanged by cell-cell contact, by free naked DNA from lysed cells, and by bacteriophages. This capacity for genetic transfer, coupled with the selective ability of antibiotics results in the presence of common genes in diverse microorganisms from different ecological and geographical niches. In sum, antibiotics select for the survivors which then thwart their efficacy. (see: Levy et al, 1999).

One approach to dealing with the aforementioned problem of bacterial resistance has been the search for new antibiotics, thereby containing the generation and spread of multidrug resistant microorganisms that have heretofore emerged. With the advent of new genetic technologies, the search for new drug targets has intensified. Another useful strategy has been the use of antimicrobial combinations as a means of increasing bactericidal action through synergistic activity of two antimicrobial agents against a particular microorganism. Combinations of antimicrobials have also been used to minimize the development of resistance. An additional approach has been to modify an existing antibiotic that is otherwise no longer suitable as an antimicrobial agent due to the development of resistance among formerly susceptible microbial populations. Such a group of antimicrobials is the cyclines.

The cyclines (sometimes referred to as tetracyclines) are broad spectrum antibiotics that bind to ribosomes and inhibit protein synthesis. These antibiotics are now restricted to treatment of infections caused by organisms of families like Chlamydia, Rickettsia, Mycoplasma and Brucella. There are two described mechanisms of tetracycline resistance: an energy dependent drug efflux system (Cohen et al, 1998; Levy, 1992; Nikaido, 1994) and ribosome protection (Burdeft, 1986).

### BRIEF SUMMARY OF THE INVENTION

In one aspect, the present invention relates to a method for treating, or protecting against, including preventing, a bacterial infection resistant to treatment with a cycline or quinolone antibiotic, comprising:

administering to an animal afflicted with, or at risk of becoming afflicted with, a cycline or quinolone resistant bacterial infection, an effective amount of said antibiotic wherein said antibiotic is complexed with a metal, such complex being either preformed or allowed to form after administration.

In a preferred embodiment, the cycline is selected from the group consisting of tetracycline, oxytetracycline, glycylcycline, doxycycline and minocycline, preferably wherein said cycline is tetracycline.

In another preferred embodiment, the antibiotic is a quinolone. The quinolone antibiotics include the subclass commonly referred to as fluoroquinolone antibiotics.

In one preferred embodiment, the metal is one of iron (II), iron (III), magnesium or calcium.

In a further preferred embodiment, the disease to be treated is caused by a bacterium of the genus Pseudomonas, Enterococcus, Staphylococcus, Streptococcus, Enterobacter, Escherichia and Klebsiella preferably from the group consisting of Pseudomonas aeruginosa, Pseudomonas putida and Pseudomonas fluorescens and most preferably Pseudomonas aeruginosa.

In preferred embodiments of the methods of the invention, the animal to be treated or protected is a mammal, especially a human patient.

### DETAILED DESCRIPTION OF THE INVENTION

This invention relates to a method of treating antibiotic, especially cycline antibiotic, resistant bacterial infections using said antibiotics in the form of a metal complex, such as metal-complexed tetracycline.

In a general aspect, the present invention relates to a method for treating or protecting against a bacterial infection resistant to treatment with an antibiotic comprising administering to an animal afflicted with, or at risk of becoming afflicted with, an antibiotic resistant infection an effective amount of the antibiotic wherein said antibiotic is complexed with a metal, either before or after administration, so that in situ complex formation is specifically contemplated.

In one aspect, the present invention relates to such treatment or protection, including prevention, where the antibiotic is a cycline. In such instance, the present invention relates to a method for treating an animal afflicted with, or protecting an animal against becoming afflicted with, a bacterial infection resistant to treatment with a cycline antibiotic, comprising exposing said animal to an effective amount of said cycline antibiotic complexed with a metal. Such protection can include complete prevention of such infection from occurring. In specific embodiments, said cycline antibiotic/metal complexed is formed in situ after administering to said animal a cycline antibiotic and a metal, either simultaneously or in sequence. In another such embodiment, the antibiotic and metal are administered as a preformed complex.

In one preferred embodiment of this method, the cycline antibiotic is selected from the group consisting of a glycylcycline, tetracycline, oxytetracycline, doxycycline and minocycline, especially tetracycline, or where the glycylcycline is tigilcycline (GAR-936) or WAY-152,288, or N,N-dimethylglycylamido derivatives of monocycline or 6-dimethyl-6-deoxytetracycline.

In another aspect, the present invention relates to such treatment or protection, including prevention, where the antibiotic is a type of quinolone. In such embodiment, the present invention relates to a method for treating an animal afflicted with, or protecting an animal against becoming afflicted with, a bacterial infection resistant to treatment with a quinolone antibiotic, comprising treating said animal with an effective amount of said quinolone antibiotic complexed with a metal. Formation of the complex in situ is specifically contemplated. Here, the antibiotic and metal may be administered separately, either simultaneously or sequentially, and the complex subsequently formed. Alternatively, the antibiotic and metal are administered as a preformed complex.

In a preferred embodiment of such method, the quinolone antibiotic is a quinolone, preferably nalidixic acid, or a fluoroquinolone, preferably a member selected from the group consisting of ciprofloxacin, trovafloxacin, grepafloxacin, levofloxacin, lomefloxacin, norfloxacin, ofloxacin, pefloxacin, sparfloxacin, gatifloxacin, moxifloxacin, gemifloxacin, and sitafloxacin.

The metals useful in the methods of the invention are commonly multivalent metals, preferably a member selected from the group consisting of iron, calcium, magnesium, zinc, manganese, copper, nickel, cobalt, and aluminum, more preferably iron (II), iron (III), magnesium or calcium.

For the antibiotic:metal complexes of the invention, such complexes preferably have a stoichiometry selected from a molecular ratio of antibiotic:metal that is 1:1, 2:1, 3:1, 4:1 or even a higher order ratio. In addition, any useful stoichiometric ratio of the antibiotics and metals recited herein may be used and stoichiometric ratio is not to be considered as a limiting factor in the methods of the invention.

The infections most commonly treated or prevented, or protected against, by the methods of the invention are those caused by a bacterium of a genus selected from the group consisting of Pseudomonas, Enterococcus, Staphylococcus, Streptococcus, Enterobacter, Escherichia and Klebsiella, preferably Pseudomonas, most preferably Pseudomonas aeruginosa, Pseudomonas putida or Pseudomonas fluorescens, especially Pseudomonas aeruginosa.

In other embodiments of the present invention, the organism is Enterococcus faecalis, Enterococcus faecium, Staphylococcus aureus, Streptococcus pneumoniae, or any coagulase negative staphylococcus.

The types of infections contemplated for treatment by the methods of the invention are any type of infection that is antibiotic resistant. In one embodiment thereof, the animal to be so protected is the victim of some form of inflammation, including burns, possibly severe burns, where infection is a potential. Thus, the infection would be associated with such burns, and the metal-complexed antibiotic, such as a metal-complexed tetracycline, could be applied topically as a suspension in a suitable carrier, including water.

In a highly preferred embodiment, the animal to be treated is a human being.

The antibiotics use to form the metal complexes useful in the methods disclosed herein include any of the different forms of such antibiotics known to medicinal chemists. Specific forms contemplated herein include, but are not limited to, any active isomers of such antibiotics, as well as salts, metabolites, polymorphs and derivatives of such antibiotics.

The metal-complexed antibiotics of the present invention are conveniently administered as a part of a composition wherein the antibiotic is suspended in a suitable pharmacological carrier. Methods well known in the art for making formulations are found in, for example, Remington: The Science and Practice of Pharmacy, (19th ed.) ed. A. R. Gennaro AR., 1995, Mack Publishing Company, Easton, Pa. Formulations for parenteral administration may, for example, contain excipients, sterile water, or saline, polyalkylene glycols such as polyethylene glycol, oils of vegetable origin, or hydrogenated napthalenes. Biocompatible, biodegradable lactide polymer, lactide/glycolide copolymer, or polyoxyethylene-polyoxypropylene copolymers may be used to control the release of the compounds. Other potentially useful parenteral delivery systems for agonists of the invention include ethylenevinyl acetate copolymer particles, osmotic pumps, implantable infusion systems, and liposomes. Formulations for inhalation may contain excipients, or example, lactose, or may be aqueous solutions containing, for example, polyoxyethylene-9-lauryl ether, glycocholate and deoxycholate, or may be oily solutions for administration in the form of nasal drops, or as a gel.

Such compositions can be utilized to combat infections in many different cases. For example, the metal-complexed antibiotic can be utilized in the form of a spray, such as by suspension in water, to prevent infection in burn victims.

All publications, patents, and patent applications cited herein are hereby incorporated by reference, as are the references cited therein. It is also to be understood that throughout this disclosure where the singular is used, the plural may be inferred and vice versa and use of either is not to be considered limiting.

The antibiotic of the invention of the invention is used in an amount effective for treating a cycline or quinolone resistant bacterial infection. In general, such amounts are:

(a) up to 4 grams per day,

(b) more preferably up to 2 grams per day

(c) more preferably up to 1 gram per day

(d) most preferably 100 mg to 1 gram per day

In carrying out the procedures of the present invention it is of course to be understood that reference to particular buffers, media, reagents, cells, culture conditions and the like are not intended to be limiting, but are to be read so as to include all related materials that one of ordinary skill in the art would recognize as being of interest or value in the particular context in which that discussion is presented. For example, it is often possible to substitute one buffer system or culture medium for another and still achieve similar, if not identical, results. Those of skill in the art will have sufficient knowledge of such systems and methodologies so as to be able, without undue experimentation, to make such substitutions as will optimally serve their purposes in using the methods and procedures disclosed herein.

The present invention will now be further described by way of the following non-limiting examples. In applying the disclosure of these examples, it should be kept clearly in mind that other and different embodiments of the present invention will no doubt suggest themselves to those of skill in the relevant art.

### EXAMPLE

Complexes of tetracycline with Fe²⁺, Fe³⁺ and Cu²⁺ were prepared. Tetracycline HCl was prepared at 1 mg/ml in water; bicarbonate@ 0.1 M pH 8.2 was prepared; salts used were ferrous ammonium sulfate, ferric chloride, copper acetate with 2.08 mmol/L HCl to keep metals in solution. Preparation of 10 mL of solutions containing 10 μg/mL tetracycline and 0.01 μmol/mL of metal cation: 9.85 mL bicarbonate solution plus 100 μl tetracycline and 50 μl of 2 μmol/mL of divalent cation. Individual components were tested for inhibition or enhancement of Pseudomonas isolated from the site. In addition, tetracycline at concentrations ranging from 0.08 μg/mL-100 μg/mL and each of the tetracycline metal complexes at 8 to 0.08 μg/mL were tested with the Pseudomonas isolate. Experimental results are presented below: (R—resistant—growth in the presence of antibiotic, I—inhibitory—minimal growth in presence of antibiotic, S—susceptible—no growth in the presence of antibiotic, Ps=Pseudomonas, the isolate being from Susquehanna flats on the Susquehanna River passing through Delaware and Maryland).

| | | | Ps |
|---|---|---|---|
| | | | isolate |
| A. | 1. a) 8 μg/mL tetracycline—8 μl stock (1 mg/mL) in 1 mL | | R |
| | TSB | | |
| | b) 1/10 0.8 μg/mL tet—8 μl of 1/10 dil′n in 1 mL TSR | | R |
| | c) 1/10 0.08 μg/mL tet—8 μl of 1/100 in 1 mL TSB | | R |
| | 2. Bicarb | 800 μl + 200 μl 5X TSB | Growth |
| | 3. Fe²⁺ 1 mL | TSB + 4 μl (4 μg/mL). | Growth |
| | 4. Fe³⁺ 1 mL | TSB + 4 μl (4 μg/mL) | Growth |
| | 5. Cu²⁺1 mL | TSB + 4 μl (4 μg/mL) | Growth |
| | 6. Tet + Fe²⁺ | (10 μg/mL tet) | |
| | | A. 80 μl + 200 μ1 5 × TSB (8 μg/mL) | S |
| | | B. 80 μl + 920 mL TSB (0.8 μg/mL) | I |
| | | C. 8 μ1 + 992 μl TSB (0.08 μg/mL) | R |
| | 7. Tet + Fe³⁺ | (10 μg/mL tet) | |
| | | A. 8 μg/mL | S |
| | | B. 0.8 μg/mL | I |
| | | C. 0.08 μg/mL | R |
| | 8. Tet + Cu²⁺ | | |
| | | A. 8 μg/mL | S |
| | | B. 0.8 μg/mL | |
| | | C. 0.08 μg/mL | R |
| B. | 1. 8 μg/mL tet | | R |
| | 0.8 μg/mL | | R |
| | 0.08 μg/mL | | R |
| | 6. Tet + Fe²⁺ | | |
| | | A. 8 μg/mL | S |
| | | B. 0.8 μg/mL | R |
| | | C. 0.08 μg/mL | R |
| | 7. Tet + Fe³⁺ | | |
| | | A. 8 μg/mL | S |
| | | B. 0.8 μg/mL | I |
| | | C. 0.08 μg/mL | R |
| | 8. Tet + Cu²⁺ | | |
| | | A. 8 μg/mL | S |
| | | B. 0.8 μg/mL | I |
| | | C. 0.08 μg/mL | R |
| C. | New Ps isolate from sediment | | |
| | 1. 8 μg/mL tet | | R |
| | 40 μg/mL | | R |
| | 100 μg/mL | | R |
| | 6. Tet + Fe²⁺ 8 μg/mL | | S |
| | | 0.8 μg/mL | I |
| | | 0.08 μg/mL | R |

| D. | 1. Tet 8 μg/mL | +4 | |
|---|---|---|---|
| | | +4 | |
| | | +4 | |
| | | +4 | |
| | 2. Bicarb | +4 | |
| | 3. Fe²⁺ | +4 | |
| | 4. Fe³⁺ | +4 | |
| | 5. Cu²⁺ | +4 | |
| | 6. Fe²⁺ + tet | 8 μg/mL | S (no growth) |
| | | 4 μg/mL | +4 |
| | | 1 μg/mL | +4 |
| | 7. Fe³⁺ + tet | 8 μg/mL | S (no growth) |
| | | 4 μg/mL | I |
| | | 1 μg/mL | +4 |
| | 8. Cu²⁺ | 8 μg/mL | S (no growth) |
| | | 4 μg/mL | I |
| | | 1 μg/mL | +4 |

What is claimed is:

1. A method for treating an animal afflicted with, or protecting an animal against becoming afflicted with, a bacterial infection resistant to treatment with a cycline antibiotic, comprising treating said animal with an effective amount of said cycline antibiotic complexed with a metal.

2. The method of claim 1 wherein said cycline antibiotic complexed with a metal is formed in situ after administering to said animal a cycline antibiotic and a metal.

3. The method of claim 2 wherein said cycline antibiotic and said metal are administered simultaneously.

4. The method of claim 1 wherein said cycline antibiotic complexed with a metal is administered to said animal as a preformed complex.

5. The method of claim 1 wherein said cycline antibiotic is selected from the group consisting of tetracycline and a glycylcycline.

6. The method of claim 1 wherein said cycline antibiotic is a member selected from the group consisting of tetracycline, oxytetracycline, doxycycline and minocycline.

7. The method of claim 6 wherein said antibiotic is tetracycline.

8. The method of claim 5 wherein said glycylcycline is tigilcycline (GAR-936), WAY-152,288 or N,N-dimethylglycylamido derivatives of monocycline or 6-dimethyl-6-deoxytetracycline.

9. The method of claim 1 wherein said metal is a multivalent metal.

10. The method of claim 1 wherein said metal is a member selected from the group consisting of iron, calcium, magnesium, zinc, manganese, copper, nickel, cobalt, and aluminum.

11. The method of claim 10 wherein said metal is a member selected from the group consisting of iron (II), iron (III), magnesium and calcium.

12. The method of claim 1 wherein said antibiotic and said metal complex in the molecular ratio 1:1, 2:1, 3:1, 4:1 or higher order ratio.

13. The method of claim 1 wherein said infection is caused by a bacterium of a genus selected from the group consisting of Pseudomonas, Enterococcus, Staphylococcus, Streptococcus, Enterobacter, Escherichia and Klebsiella.

14. The method of claim 13 wherein said genus is Pseudomonas.

15. The method of claim 14 wherein said bacterium is a member selected from the group consisting of Pseudomonas aeruginosa, Pseudomonas putida and Pseudomonas fluorescens.

16. The method of claim 11 wherein said bacterium is Pseudomonas aeruginosa.

17. The method of claim 13 wherein said organism is a member selected from the group consisting of Enterococcus faecalis, Enterococcus faecium, Staphylococcus aureus, Streptococcus pneumoniae, and coagulase negative staphylococcus.

18. The method of claim 1 wherein said infection is associated with a skin burn.

19. The method of claim 18 wherein said metal-complexed cycline is applied topically.

20. The method of claim 18 wherein said animal is a human being.

21. A method for treating an animal afflicted with, or protecting an animal against becoming afflicted with, a bacterial infection resistant to treatment with a quinolone antibiotic, comprising treating said animal with an effective amount of said quinolone antibiotic complexed with a metal.

22. The method of claim 21 wherein said quinolone antibiotic complexed with a metal is formed in situ after administering to said animal a quinolone antibiotic and a metal.

23. The method of claim 22 wherein said quinolone antibiotic and said metal are administered simultaneously.

24. The method of claim 21 wherein said quinolone antibiotic complexed with a metal is administered to said animal as a preformed complex.

25. The method of claim 21 wherein said quinolone antibiotic is a fluoroquinolone.

26. The method of claim 25 wherein said quinolone is nalidixic acid.

27. The method of claim 25 wherein said fluoroquinolone is a member selected from the group consisting of ciprofloxacin, trovafloxacin, grepafloxacin, levofloxacin, lomefloxacin, norfloxacin, ofloxacin, pefloxacin, sparfloxacin, gatifloxacin, moxifloxacin, gemifloxacin, and sitafloxacin.

28. The method of claim 21 wherein said metal is a multivalent metal.

29. The method of claim 21 wherein said metal is a member selected from the group consisting of iron, calcium, magnesium, zinc, manganese, copper, nickel, cobalt, and aluminum.

30. The method of claim 29 wherein said metal is a member selected from the group consisting of iron (II), iron (III), magnesium and calcium.

31. The method of claim 21 wherein said antibiotic and said metal complex in the molecular ratio 1:1, 2:1, 3:1, 4:1 or higher order ratio.

32. The method of claim 21 wherein said infection is caused by a bacterium of a genus selected from the group consisting of Pseudomonas, Enterococcus, Staphylococcus, Streptococcus, Enterobacter, Escherichia and Klebsiella.

33. The method of claim 32 wherein said genus is Pseudomonas.

34. The method of claim 33 wherein said bacterium is a member selected from the group consisting of Pseudomonas aeruginosa, Pseudomonas putida and Pseudomonas fluorescens.

35. The method of claim 30 wherein said bacterium is Pseudomonas aeruginosa.

36. The method of claim 32 wherein said organism is a member selected from the group consisting of Enterococcus faecalis, Enterococcus faecium, Staphylococcus aureus, Streptococcus pneumoniae, and coagulase negative staphylococcus.

37. The method of claim 21 wherein said infection is associated with a skin burn.

38. The method of claim 37 wherein said metal-complexed quinolone is applied topically.

39. The method of claim 37 wherein said animal is a human being.
